(19) United States
(12) Patent Application Publication
(10) Pub. No.: US 2022/0009892 A1
(43) Pub. Date: Jan. 13, 2022

(54) SUBSTITUTED PYRIDINE CARBOXYLIC ACIDS, THEIR PREPARATION METHOD AND COMPOSITIONS THEREOF

(71) Applicant: INCILIA THERAPEUTICS PRIVATE LIMITED, Hyderabad, Telangana (IN)

(72) Inventors: Vijay JOGUPARTHI, Hyderabad (IN); Narendra Varma GADDIRAJU, Hyderabad (IN); Pedda Obireddygari VENKATARAMANA REDDY, Hyderabad (IN)

(73) Assignee: INCILIA THERAPEUTICS PRIVATE LIMITED, Hyderabad, Telangana (IN)

(21) Appl. No.: 17/293,874

(22) PCT Filed: Nov. 16, 2019
(86) PCT No.: PCT/IN2019/050849

§ 371 Date: May 13, 2021

(30) Foreign Application Priority Data
Nov. 16, 2018 (IN) 201841043166

(51) Int. Cl. C07D 213/80; C07D 401/12

(52) U.S. Cl. CPC C07D 213/80; C07D 401/12

(57) ABSTRACT

The present invention relates to novel substituted pyridine carboxylic acid derivatives and their preparation methods. This invention is also directed to pharmaceutical compositions containing such compounds and combinations thereof with at least one therapeutic agent. These substituted pyridine carboxylic acid compounds and their pharmaceutical acceptable salts and esters are useful in the treatment or control of various metabolic disorders.

### CROSS REFERENCE

This application claims priority from Indian Patent Application No. 201841043166 filed on Nov. 16, 2018.

### FIELD OF INVENTION

The present invention relates to novel substituted pyridine carboxylic acid derivatives and their preparation methods. These compounds and their pharmaceutical acceptable salts and esters are useful in the treatment or control of various metabolic disorders. This invention is also directed to pharmaceutical compositions containing such compounds and combinations thereof with at least one therapeutic agent.

### BACKGROUND

The pyridine derivatives have wider therapeutic application for treatment of various disorders and are being explored for various new activities.

U.S. Pat. No. 3,655,679 discloses aryl pyridine carboxylic acids and their derivatives which exhibit anti-inflammatory, analgesic properties.

European patent publication no. EP0109027A1 discloses 2-alkoxy-5-(pyridinyl) pyridine derivatives and their use as cardiotonics.

US20040081672 A1 publication relates to application of niacinamide, niacin, and niacin esters derivatives of skin beneficial organic acids for the Synergistic treatment or prevention of topical disorders of Skin Such as acne, rosacea, skin wrinkles, age-spots, canker sores, striae distensae, pimples, and skin redness.

WO2005075464 A1, discloses pyridine derivatives for treatment of pain mediated by cannabinoid 2 receptor. U.S. Pat. No 6,656,957 discloses halo-substituted pyridine derivatives with activity for modulating glutamatergic signal transmission.

Despite several options available, there remains an unmet need to develop a most suitable pharmaceutical compound of substituted pyridine carboxylic acid derivatives and compositions thereof. Therefore there is need for development of novel pyridine derivatives with various therapeutic activities. The present inventors have developed specific novel pyridine derivatives with a good yield and purity using simple and economical process with varying activity in field of pharmaceutical and medicinal chemistry.

### SUMMARY

The present invention relates to substituted pyridine carboxylic acid derivative compounds for managing cardiovascular diseases, inflammatory diseases, diabetes, cancer, nutritional disorders and dermatological conditions along with other metabolic disorders.

Specifically, it is an object of the present invention to provide a compound of general formula (I), or a pharmaceutically acceptable salt, isomer, ester, prodrug or solvate thereof:

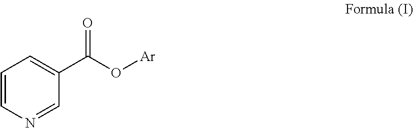

wherein, Ar is selected from the following compounds as given below:

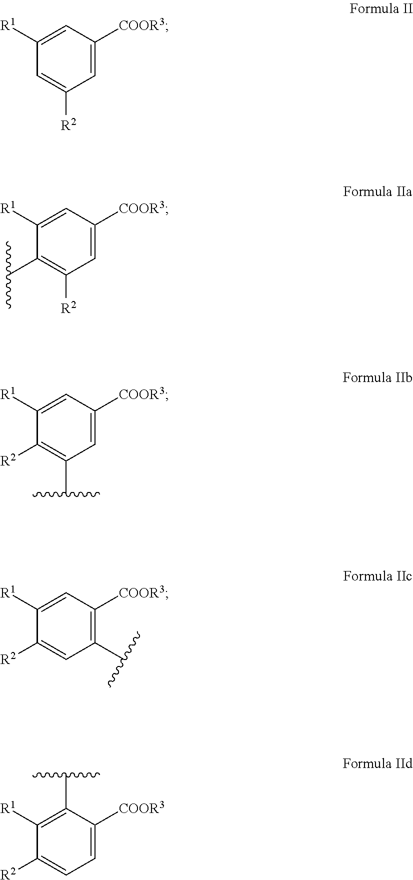

and
R¹ represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group;
R² represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group;
R³ represents one or more independent substitutions in the benzene moiety selected from the group comprising of —H, -alkyl, -amine, phenyl, benzene or any other suitable aliphatic or aromatic group;

and wherein R¹ and R² could be the same or different functional moieties selected from the above functional groups, and the position of R¹ and R² can be interchangeable used.

A further object of the present invention is to provide a simple and economical process for the preparation of the compound of formula (I).

An another object of the present invention is to provide a pharmaceutical compositions comprising a pharmaceutically effective amount of the compound of formula (I) or a pharmaceutically acceptable salt, isomer, ester, prodrug or solvate thereof and a pharmaceutically acceptable carrier or excipient.

A further object of the invention is to provide a combination composition comprising a pharmaceutically effective amount of the compound of formula (I) or a pharmaceutically acceptable salt, isomer, ester, prodrug or solvate thereof and a pharmaceutically acceptable carrier or excipient in combination with another therapeutic agent.

### BRIEF DESCRIPTION OF FIGURES

FIG. 1 depicts a ¹H NMR spectrum of Compound of Formula (I), particularly of INL3001115.

FIG. 2 depicts a ¹³C NMR spectrum of Compound of Formula (I), particularly of INL3001115.

FIG. 3 depicts a FT-IR spectrum of Compound of Formula (I), particularly of INL3001115.

FIG. 4 depicts a Mass spectrum of Compound of Formula (I), particularly of INL3001115.

FIG. 5 depicts a ¹H NMR spectrum of Compound of Formula (I), particularly of INL3001136.

FIG. 6 depicts a ¹³C NMR spectrum of Compound of Formula (I), particularly of INL3001136.

FIG. 7 depicts a FT-IR spectrum of Compound of Formula (I), particularly of INL3001136.

FIG. 8 depicts a Mass spectrum of Compound of Formula (I), particularly of INL3001136.

FIG. 9 depicts a ¹H NMR spectrum of Compound of Formula (I), particularly of INL3001119.

FIG. 10 depicts a ¹³C NMR spectrum of Compound of Formula (I), particularly of INL3001119.

FIG. 11 depicts a FT-IR spectrum of Compound of Formula (I), particularly of INL3001119.

FIG. 12 depicts a Mass spectrum of Compound of Formula (I), particularly of INL3001119.

FIG. 13 depicts a ¹H NMR spectrum of Compound of Formula (I), particularly of INL3001117.

FIG. 14 depicts a ¹³C NMR spectrum of Compound of Formula (I), particularly of INL3001117.

FIG. 15 depicts a FT-IR spectrum of Compound of Formula (I), particularly of INL3001117.

FIG. 16 depicts a Mass spectrum of Compound of Formula (I), particularly of INL3001117.

FIG. 17 depicts a FT-IR spectrum of Compound of Formula (I), particularly of INL3001101.

FIG. 18 depicts a Mass spectrum of Compound of Formula (I), particularly of INL3001101.

FIG. 19 depicts a synthesis scheme for INL3001117.

FIG. 20 depicts a synthesis scheme for INL3001119.

### DETAILED DESCRIPTION

The following paragraphs detail various embodiments of the invention. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and combined with other important aspects of the invention described elsewhere within this specification as a whole, and including the examples and figures. The skilled person will appreciate that the invention extends to such combinations of features and that these have not been recited in detail here in the interests of brevity.

The present invention relates to a substituted pyridine derivative compound of general formula (I), or a pharmaceutically acceptable salt, isomer, ester, prodrug or solvate thereof:

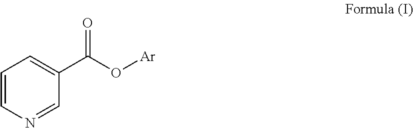

wherein, Ar is selected from the following compounds as given below:

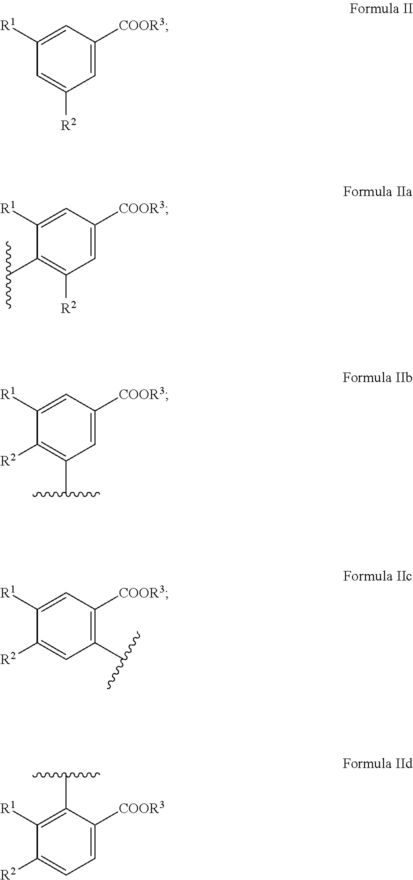

and
R¹ represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group;
R² represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group;
R³ represents one or more independent substitutions in the benzene moiety selected from the group comprising of —H, -alkyl, -amine, phenyl, benzene or any other suitable aliphatic or aromatic group;

and wherein R¹ and R² could be the same or different functional moieties selected from the above functional groups, and the position of R¹ and R² can be interchangeable used.

In another preferred embodiment the present invention relates to a process for preparation of the compound of formula (I), its isolation and characterisation.

In another preferred embodiment the present invention provides a combination composition comprising a pharmaceutically effective amount of the compound of formula (I) or a pharmaceutically acceptable salt, isomer, ester, prodrug or solvate thereof and a pharmaceutically acceptable carrier or excipient in combination with another therapeutic agent.

In yet another preferred embodiment the present invention provides a pharmaceutical compositions useful for treatment of cholinergic receptor mediated diseases, wherein the composition comprises a pharmaceutically effective amount of the compound of formula (I) or a pharmaceutically acceptable salt, isomer, ester, prodrug or solvate thereof and a pharmaceutically acceptable carrier or excipient.

In another embodiment the compounds of the present invention are used in the management of hyperlipidemia and hypercholesterolemia and associated cardiovascular diseases, diabetes, nutritional disorders, inflammation, proliferative disease, skin disorders and other metabolic disorders.

In some embodiments, compositions of the invention are in the form of solid, liquid or semisolid dosage forms for oral, topical, rectal, intravenous, intramuscular administrations.

Non-limiting examples of suitable solid dosage forms include tablets (e.g. suspension tablets, bite suspension tablets, rapid dispersion tablets, chewable tablets, melt tablets, effervescent tablets, bi-layer tablets, etc), caplets, capsules (e.g. a soft or a hard gelatin capsule filled with solid andor liquids), powder (e.g. a packaged powder, a dispensable powder or an effervescent powder), lozenges, sachets, cachets, troches, pellets, granules, micro-granules, encapsulated micro-granules, powder aerosol formulations, or any other solid dosage form reasonably adapted for oral administration.

Non-limiting examples of suitable liquid dosage forms include solutions, suspension, elixirs, syrups, liquid aerosol formulations, etc.

Non-limiting examples of suitable semi-solid dosage forms include ointment, gel, emulsions and creams, etc.

In further embodiment the compounds according to the invention can be converted to various administration forms. This can be done in a manner known per se, by mixing with inert, nontoxic, pharmaceutically suitable excipients. These excipients include disintegrants, binders, carriers, solvents, emulsifiers and dispersing or wetting agents, surfactants, lubricants, glidants, synthetic and natural polymers, stabilizers, dyes, flavour andor odour correctants.

In the present invention the following terms have the meaning detailed below.

The term “salt” must be understood as any form of a compound used in accordance with this invention in which said compound is in ionic form or is charged and coupled to a counter-ion (a cation or anion) or is in solution. This definition also includes quaternary ammonium salts and complexes of the molecule with other molecules and ions, particularly, complexes formed via ionic interactions. The definition includes in particular physiologically acceptable salts; this term must be understood as equivalent to “pharmacologically acceptable salts” or “pharmaceutically acceptable salts”.

The term “pharmaceutically acceptable salts” in the context of this invention means any salt that is tolerated physiologically (normally meaning that it is not toxic, particularly, as a result of the counter-ion) when used in an appropriate manner for a treatment, applied or used, particularly, in humans andor mammals. Non-limiting examples of the salts include non-toxic, inorganic and organic base or acid addition salts of compounds of the present invention. In many cases, the compounds of the present invention are capable of forming acid andor base salts by virtue of the presence of amino andor carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stochiometric amount of the appropriate base (such as Na, Ca, Mg, or K hydroxide, carbonate, bicarbonate, or the like), or by reacting free base forms of these compounds with a stochiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

The term “solvate” in accordance with this invention should be understood as meaning any form of the compound in accordance with the invention in which said compound is bonded by a non-covalent bond to another molecule (normally a polar solvent), including especially hydrates and alcoholates. A preferred solvate is the hydrate.

The term “pharmaceutically-acceptable carrier”, as used herein, means one or more compatible solid or liquid filler diluents or all solvents, dispersion media, coatings, surfactants, antioxidants, preservatives, isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (refer, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

Any compound that is a prodrug of a compound of formula (I) is also within the scope of the invention. The term “prodrug” is used in its broadest sense and encompasses those derivatives that are converted in vivo to the compounds of the invention. Examples of prodrugs include, but are not limited to, derivatives and metabolites of the compounds of formula (I). Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6th ed. (Donald J. Abraham ed., 2001, Wiley) and “Design and Applications of Prodrugs” (H. Bundgaard ed., 1985, Harwood Academic Publishers).

The term “therapeutically effective amount” of a compound of the present invention refers to an amount of the compound of the present invention that will elicit the biological or medical response of a subject, or ameliorate symptoms, slow or delay disease progression, or prevent a disease, etc.

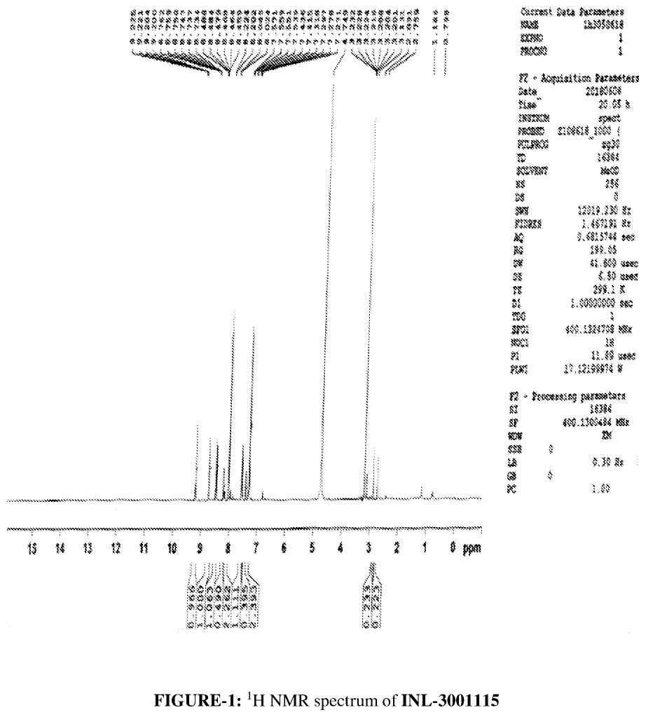

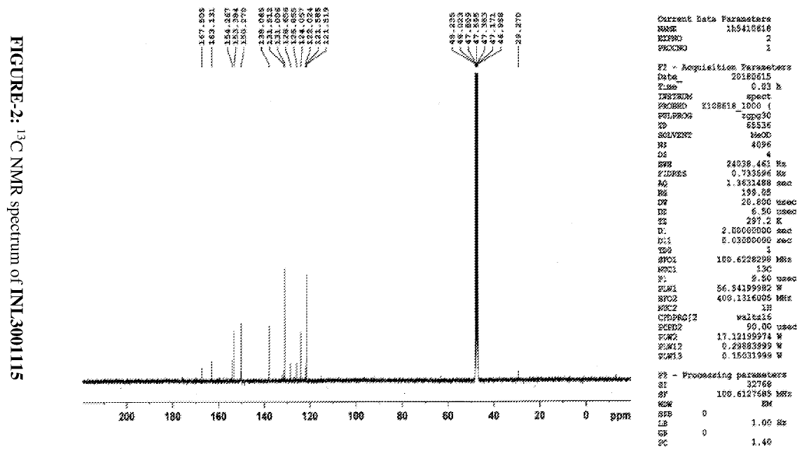

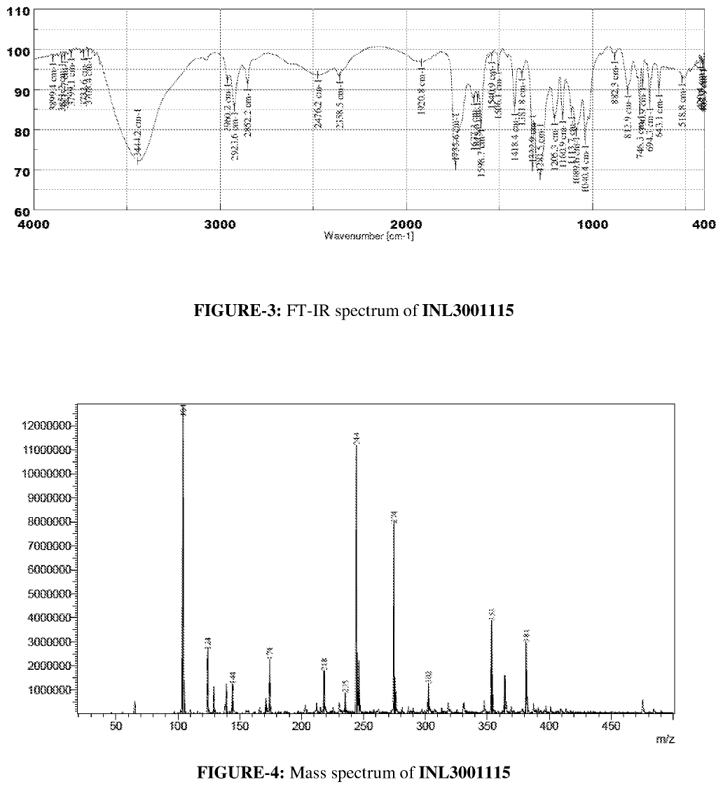

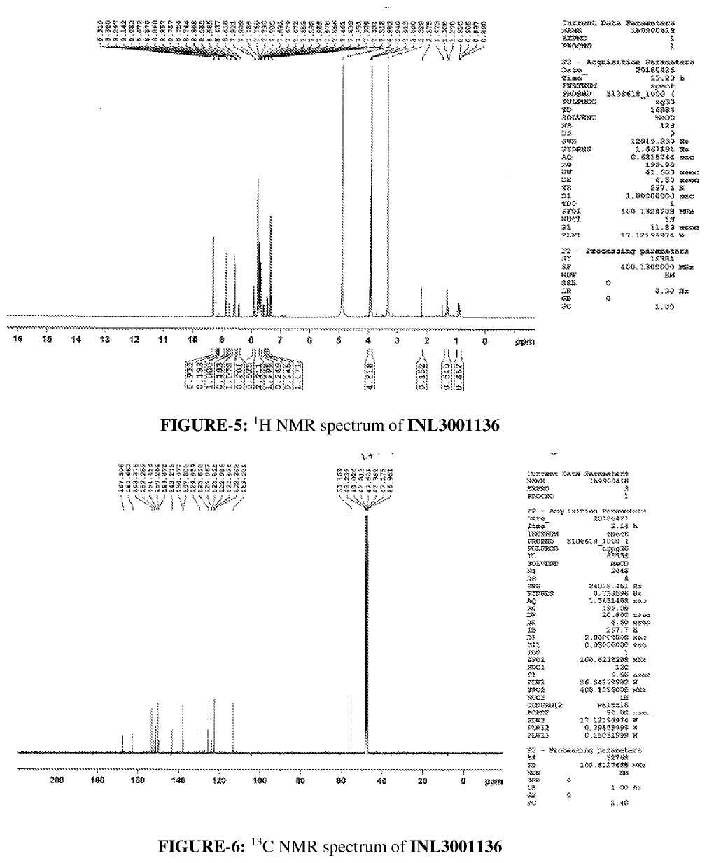

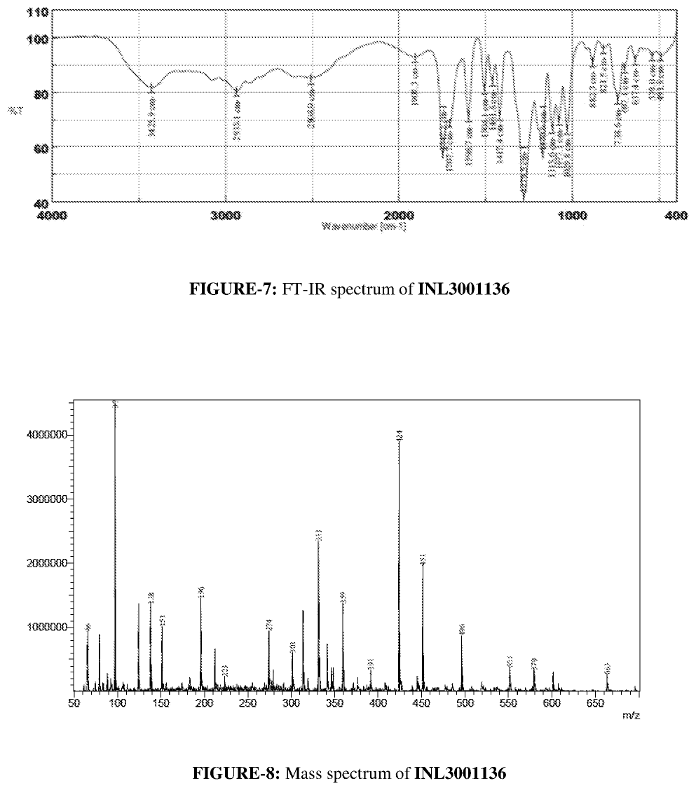

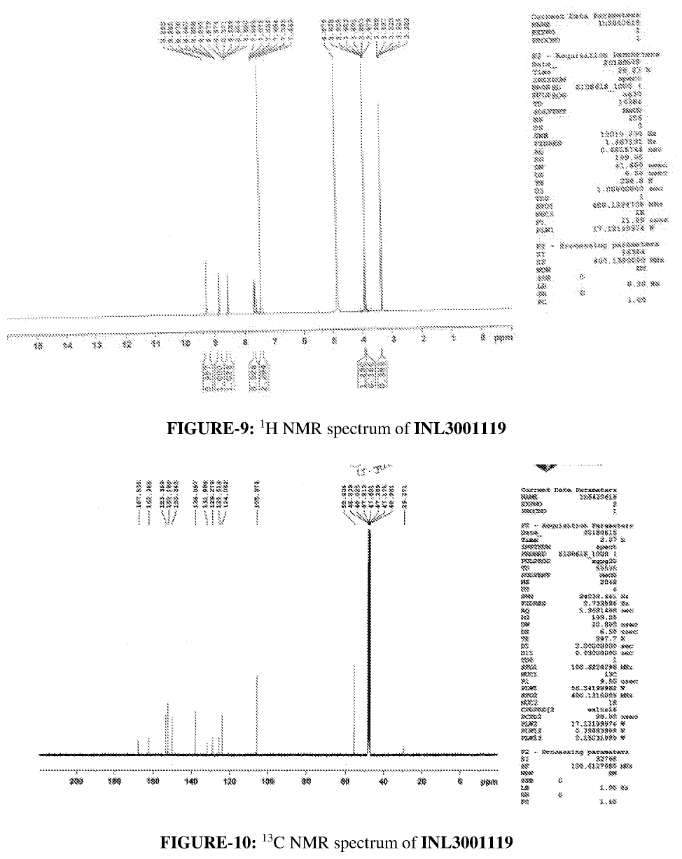

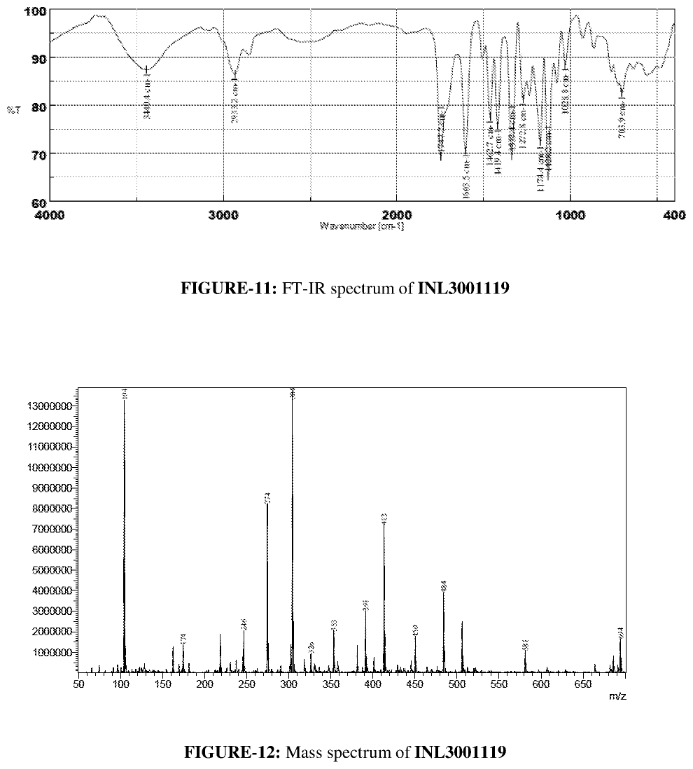

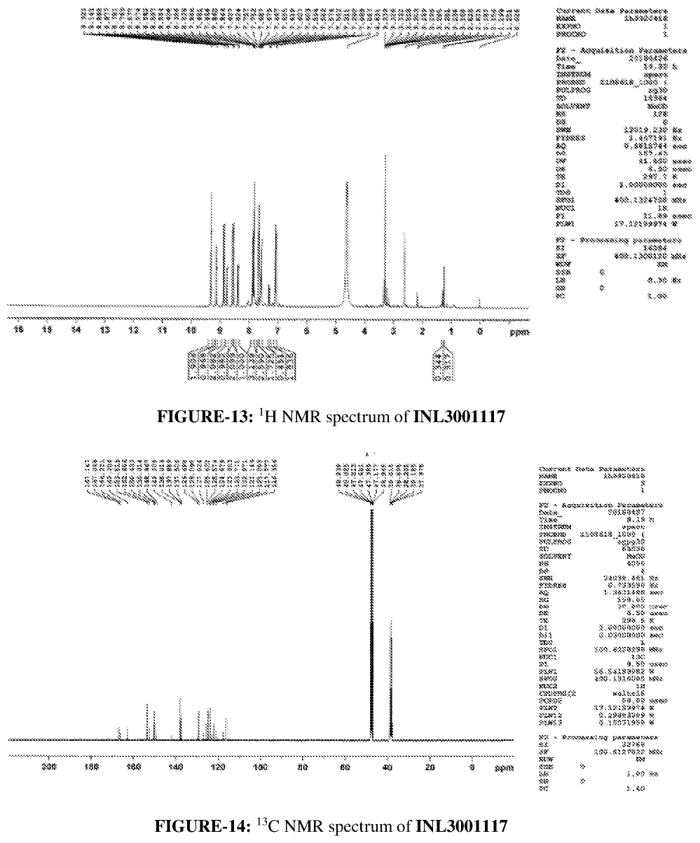

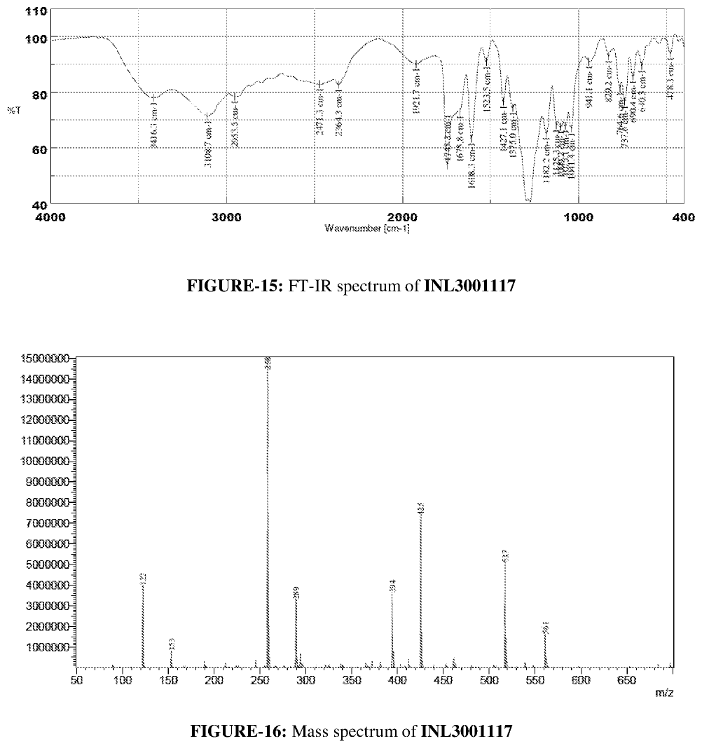

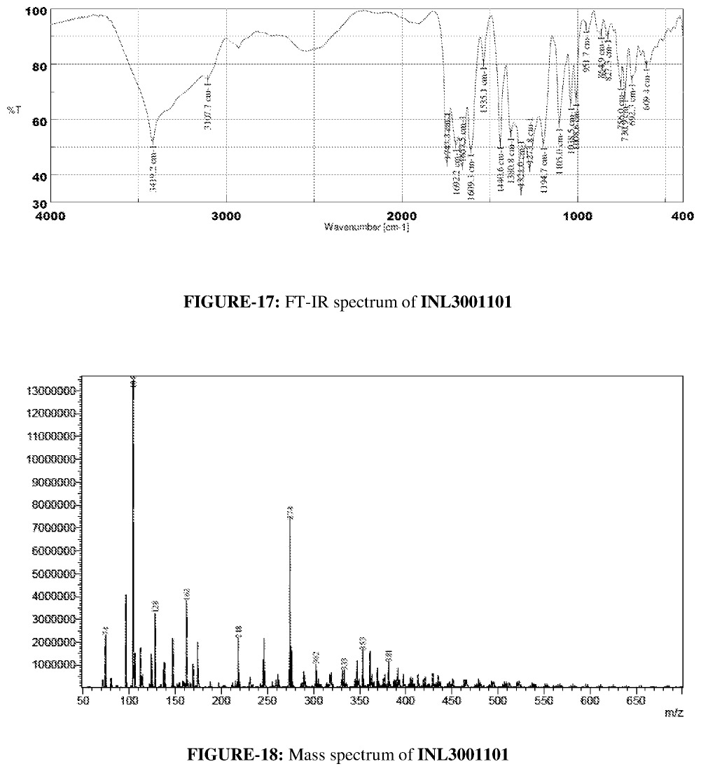

FIGS. 1-18, briefly characterise some of the non-limiting substituted pyridine derivative compound of general formula (I), or a pharmaceutically acceptable salt, isomer, ester, prodrug or solvate thereof:

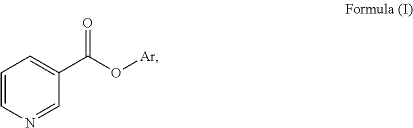

viz., INL3001115, INL3001136, INL3001119, INL3001117, INL3001101 by means of Mass, FT-IR and NMR spectra of the novel substituted pyridine derivative compounds.

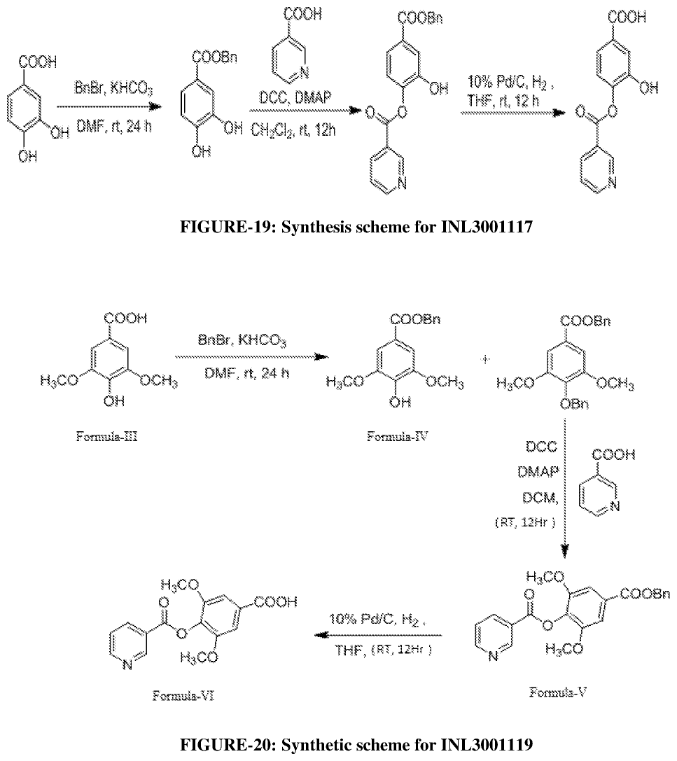

FIG. 19-20, brief a schematic synthetic process for preparation of the compound INL3001117, INL3001119.

In another embodiment of the invention the present invention relates to a process for preparation of compound of Formula (I), wherein the process step avoids multiple purification and extraction steps as used in conventional synthetic process there by reducing the overall process cost and also prevents multiple solvent washing.

In yet another embodiment the residues obtained in the process steps for preparation of compound of formula (I) can be used for the next step with or without any additional purification steps.

In another embodiment the process includes various non-limiting reaction components like chlorinating agent, organic and inorganic solvent, heterocyclic aromatic compounds, polar and non-polar solvents, crystallizing agents or their mixtures.

The following preparation process as set forth to aid in an understanding of the invention, and is not intended, and should not be construed, to limit in any way the scope of the invention. A person skilled in the art will readily recognize the various modifications and variations that may be performed without altering the scope of the present invention. Such modifications and variations are encompassed within the scope of the invention and the examples do not in any way limit the scope of the invention.

### EXAMPLE

### Example-1

### Procedure for Synthesis of Substituted Pyridine Carboxylic Acid Derivatives of Formula (I)

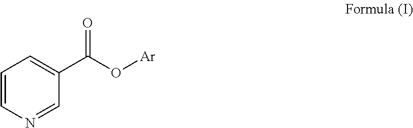 [0072] A pyridine carboxylic acid was taken into a flask and to it a suitable chlorinating agent was added.
The resultant mixture was then stirred at a higher temperature for sufficient time.
Then the excess chlorinating agent was removed from the reaction mixture and the obtained residue was used for the next reaction step with or without purification.
Thereafter a solution of the above residue was prepared in a solvent to which a heterocyclic aromatic organic compound was added followed by addition of a solution of phenolic derivative.
The resulting mixture was stirred above room temperature for sufficient time.
The reaction was monitored for completion, which was confirmed by TLC or any other suitable known analytical technique.
Then the reaction mixture was cooled and quenched with polar solvent.
The solvent was completely removed by using aromatic hydrocarbon under rota evaporator.
Thereafter the residue was dissolved in alcohol or any other organic solvent and silica gel was added to it followed by drying to get dried slurry.
The slurry was then loaded onto column and eluted using suitable solvent to get the pyridine derivative compound with good yield.
Thereafter the isolated compound may be subjected to crystallization by dissolving in organic solvent and adding a good crystallization solvent, followed by cooling to get the crystal and separating the crystal from mother liquor and drying.

### Example-2

### Procedure for Synthesis of Substituted Pyridine Carboxylic Acid Derivatives:

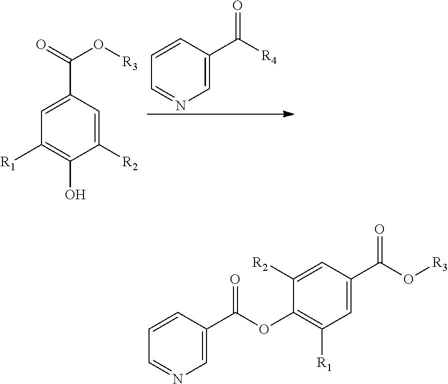

Wherein R¹ & R² represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group; R³ represents one or more independent substitutions in the benzene moiety selected from the group comprising of —H, -alkyl, -amine, phenyl, benzene or any other suitable aliphatic or aromatic group; and wherein R¹ and R² could be the same or different functional moieties selected from the above functional groups, and the position of R¹ and R² can be interchangeable used.

Experimental procedure: A solution of nicotinic acid derivative was reacted with corresponding solution of hydroxy substituted benzoate derivatives followed by stirring the mixture above room temperature or at elevated temperature, with continuous monitoring for completion, and confirmation by TLC or any other suitable known analytical technique. The reaction mixture was cooled and quenched with solvent, and the solvent was completely removed under rota evaporator or by using precipitation technique. The residue was dissolved in alcohol or any other organic solvent and silica gel was added to it followed by drying to get dried slurry. Then the slurry was loaded onto column and eluted using suitable solvent to get the pyridine derivative compound with good yield. The isolated compound may be subjected to crystallization by dissolving in organic solvent and adding a good crystallization solvent, followed by cooling to get the crystal and separating the crystal from mother liquor and drying.

### Example 3

### Synthesis of Carbomethoxy Aryl Nicotinates

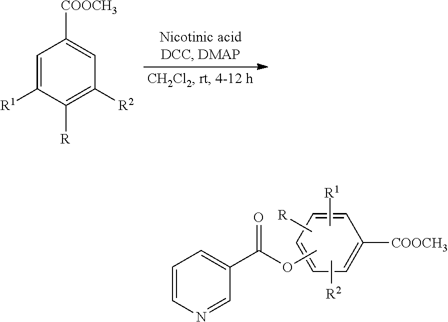

Wherein,
R═OH, R¹, R²═H
R═OH, R¹═OH, R²═H,
R═H, R¹, R²═OH,
R═OH, R¹═OH, R²═H,
R═OH, R¹═OCH₃, R²═H,
R═OH, R¹═OCH₃, R²═OCH₃.

A mixture of phenolic methyl ester derivative (1 eq.), nicotinic acid (1.5 eq.), N,N′-Dicyclo-hexyl-carbodiimide (DCC) (1.5 eq.), 4-Dimethylaminopyridine (DMAP) (0.1 eq.) and dichloromethane (10 wv) was stirred at ambient temperature for 6-12 Hr. Progress of the reaction was monitored by thin layer chromatography. Upon completion of the reaction, reaction mixture was filtered and the dichloromethane layer was stirred with saturated NaHCO₃ solution and separated. The separated organic layer was distilled off under vacuum, followed by finally collecting the crude product and subjecting it to column chromatography with 15-30% ethylacetate in n-hexane as eluent to get pure target compounds in 50-85% yields.

### Example 3.1

### Synthesis of 4-(methoxycarbonyl)phenyl nicotinate

The process of synthesis same as in example 3 was used for preparation of 4-(methoxycarbonyl)phenyl nicotinate in a yield of 85%.

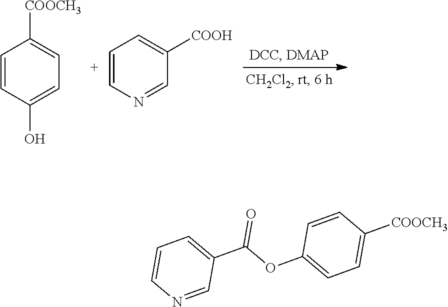 [SMILES: COC(=O)c1ccc(O)cc1 ; O=C(O)c1cccnc1 ; COC(=O)c1ccc(OC(=O)c2cccnc2)cc1]

Characterisation (Analytical data): Yield: 85%; Mp: 127-128; ¹H NMR (400 MHz, DMSO-d₆) δ 9.28 (d, J=1.6 Hz, 1H), 8.92 (dd, J=4.8, 1.6 Hz, 1H), 8.48 (dt, J=8.0, 1.9 Hz, 1H), 8.08 (d, J=8.7 Hz, 2H), 7.67 (dd, J=7.9, 4.9 Hz, 1H), 7.51 (d, J=8.7 Hz, 2H), 3.88 (s, 3H); ¹³C NMR (100 MHz, DMSO-d₆) δ 166.0, 163.7, 154.8, 154.5, 151.1, 138.0, 131.3, 128.0, 125.3, 124.5, 122.8, 52.7.

### Example 3.2

### Synthesis of 2-methoxy-4-(methoxycarbonyl)phenyl nicotinate

The process of synthesis same as in example 3 was used for preparation of 2-methoxy-4-(methoxycarbonyl)phenyl nicotinate in a yield of 82%.

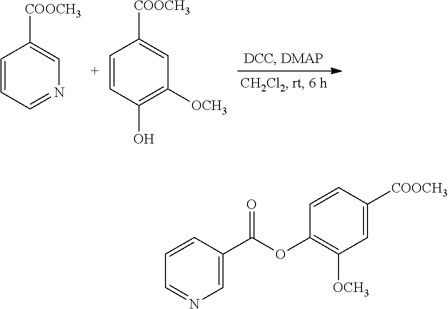 [SMILES: COC(=O)c1cccnc1 ; COC(=O)c1ccc(O)c(OC)c1 ; COC(=O)c1ccc(OC(=O)c2cccnc2)c(OC)c1]

Characterisation (Analytical data): Yield: 82%; ¹H NMR (400 MHz, DMSO-d₆) δ 9.26 (dd, J=2.3, 0.9 Hz, 1H), 8.93 (dd, J=4.9, 1.7 Hz, 1H), 8.48 (dt, J=8.0, 1.9 Hz, 1H), 7.69-7.65 (m, 3H), 7.47 (d, J=8.0 Hz, 1H), 3.89 (s, 3H), 3.86 (s, 3H). ¹³C NMR (100 MHz, DMSO-d₆) δ 166.0, 163.1, 155.0, 151.4, 151.0, 143.4, 138.1, 129.3, 124.9, 124.7, 123.9, 122.6, 113.6, 56.6, 52.8; MS (ESI): mlz calculated for C₁₅H₁₃NO₅: 287.08; found: 288 [M+H]⁺.

### Example 3.3

### Synthesis of 2,6-dimethoxy-4-(methoxycarbonyl)phenyl nicotinate

The process of synthesis same as in example 3 was used for preparation of 2,6-dimethoxy-4-(methoxycarbonyl)phenyl nicotinate in a yield of 80%.

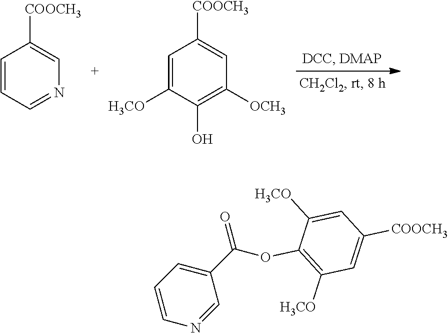 [SMILES: COC(=O)c1cccnc1 ; COC(=O)c1cc(OC)c(O)c(OC)c1 ; COC(=O)c1cc(OC)c(OC(=O)c2cccnc2)c(OC)c1]

Characterisation (Analytical data): Yield: 80%; Mp: 173-175° C.; ¹H NMR (400 MHz, DMSO-d₆) δ 9.26 (dd, J=2.2, 0.8 Hz, 1H), 8.94 (dd, J=4.8, 1.7 Hz, 1H), 8.48 (dt, J=8.0, 1.8 Hz, 1H), 7.69-7.65 (m, J=8.0, 4.9, 0.8 Hz, 1H), 7.37 (s, 2H), 3.90 (s, 3H), 3.85 (s, 6H); ¹³C NMR (100 MHz, DMSO-d₆) δ 166.0, 162.7, 155.1, 152.3, 151.0, 138.1, 131.9, 128.7, 124.8, 124.6, 106.3, 56.8, 52.9; IR (KBr, v^{˜}, cm⁻¹) 3075, 2962, 2933, 2843, 1745, 1715, 1602, 1504, 1459, 1414, 1342, 1253, 1189, 1068, 1019, 993, 883, 756, 727, 698; MS (ESI): m/z calculated for C₁₆H₁₅NO₆: 317.09; found: 318 [M+H]⁺.

### Example 3.4

### Synthesis of 4-(methoxycarbonyl)-1,2-phenylene dinicotinate

The process of synthesis same as in example 3 was used for preparation of 4-(methoxycarbonyl)-1,2-phenylene dinicotinate in a yield of 76%.

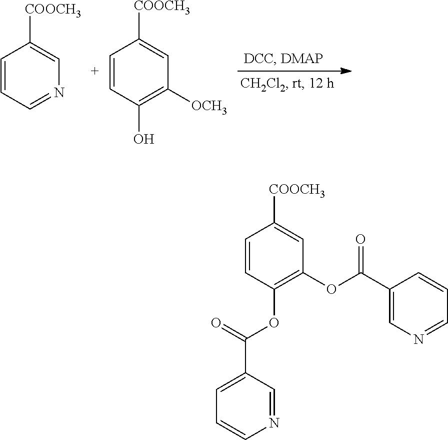 [SMILES: COC(=O)c1cccnc1 ; COC(=O)c1ccc(O)c(OC)c1 ; COC(=O)c1ccc(OC(=O)c2cccnc2)c(OC(=O)c2cccnc2)c1]

2.2 eq. of nicotinic acid used instead of 1.5 eq., for the above reaction.

Characterisation (Analytical data): Yield: 76%; Mp: 159-161° C.; ¹H NMR (400 MHz, DMSO-d₆) δ 9.15-9.08 (m, 2H), 8.83 (td, J=4.7, 1.6 Hz, 2H), 8.36-8.32 (m,2H), 8.17 (d, J=2.0 Hz, 1H), 8.06 (dd, J=8.5, 2.1 Hz, 1H), 7.76 (d, J=8.5 Hz, 1H), 7.59-7.52 (m, 2H), 3.91 (s, 3H); ¹³C NMR (100 MHz, DMSO-d₆) δ 165.3, 162.9, 162.7, 155.1, 155.0, 150.9, 150.8, 146.0, 142.2, 137.9, 137.8, 129.1, 128.7, 125.3, 124.8, 124.6, 124.6, 124.5, 53.0; IR (KBr, v^{˜}, cm⁻¹) 3084, 3011, 1750, 1719, 1641, 1596, 1404, 1284, 1188, 1094, 1022, 730, 619.

### Example 4

### Synthesis of 2-hydroxy-4-(methoxycarbonyl)phenyl nicotinate

### Step-1: Synthesis of 2-(benzyloxy)-4-(methoxycarbonyl)phenyl nicotinate (Process of Synthesis as in Example 3)

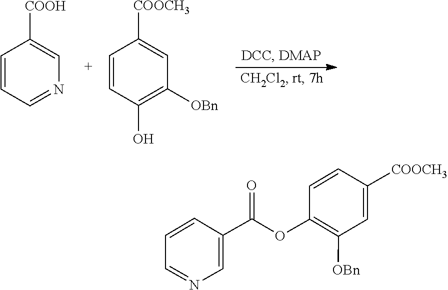 [SMILES: O=C(O)c1cccnc1 ; COC(=O)c1ccc(O)c(OCc2ccccc2)c1 ; COC(=O)c1ccc(OC(=O)c2cccnc2)c(OCc2ccccc2)c1]

Characterisation (Analytical data): Yield: 75%; ¹H NMR (400 MHz, DMSO-d₆) δ 9.26 (dd, J=2.2, 0.8 Hz, 1H), 8.92 (dd, J=4.8, 1.7 Hz, 1H), 8.47 (dt, J=8.0, 1.8 Hz, 1H), 7.78 (d, J=1.9 Hz, 1H), 7.69 (dd, J=8.2, 1.9 Hz, 1H), 7.67-7.64 (m, 1H), 7.51 (d, J=8.3 Hz, 1H), 7.32 (dd, J=7.3, 2.4 Hz, 2H), 7.29-7.24 (m, 3H), 5.24 (s, 2H), 3.88 (s, 3H).

### Step-2: Synthesis of 2-hydroxy-4-(methoxycarbonyl)phenyl nicotinate

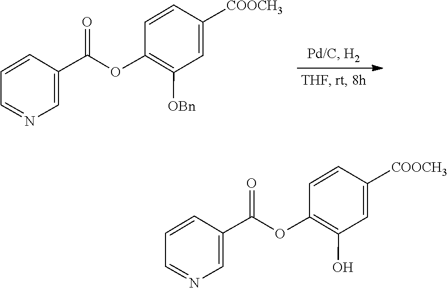 [SMILES: COC(=O)c1ccc(OC(=O)c2cccnc2)c(OCc2ccccc2)c1 ; COC(=O)c1ccc(OC(=O)c2cccnc2)c(O)c1]

In a two neck round bottom flask, 2-(benzyloxy)-4-(methoxycarbonyl)phenyl nicotinate (0.5 g, 1 eq.), Tetrahydrofuran (THF) (10 mL), and 10% PdC (0.1 g) were mixed. Then the flask was capped with a septum, nitrogen gas, vacuum was applied and released. Later, the flask was filled with hydrogen gas using balloon. The suspension was stirred for 4 hr at room temperature. Complete conversion of the starting material was confirmed by thin layer chromatography. Upon completion of the reaction, PdC was filtered on celite bed and washed with THF. Finally, THF was evaporated under vacuum and crude was stirred in dichloromethane to afford debenzylated products as white solids.

Characterisation (Analytical data): Yield: 55%; ¹H NMR (400 MHz, DMSO-d₆) δ 10.84 (s, 1H), 9.29-9.24 (m, 1H), 8.91 (dd, J=4.8, 1.6 Hz, 1H), 8.47 (dt, J=7.9, 1.9 Hz, 1H), 7.80 (dd, J=6.5, 2.1 Hz, 2H), 7.66 (dd, J=7.8, 4.9 Hz, 1H), 7.09 (d, J=9.1 Hz, 1H), 3.82 (s, 3H).

### Example 5

### Synthesis of 2-hydroxy-5-(methoxycarbonyl)phenyl nicotinate

### Step-1: Synthesis of 2-((tert-butyldimethylsilyl)oxy)-5-(methoxycarbonyl)phenyl nicotinate

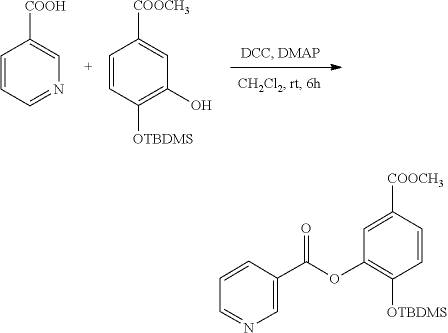 [SMILES: O=C(O)c1cccnc1 ; COC(=O)c1ccc(O[Si](C)(C)C(C)(C)C)c(O)c1 ; COC(=O)c1ccc(O[Si](C)(C)C(C)(C)C)c(OC(=O)c2cccnc2)c1]

A mixture of methyl 4-((tert-butyldimethylsilyl)oxy)-3-hydroxybenzoate (1 g, 1 eq.), nicotinic acid (0.65 g, 1.5 eq.), N,N′-Dicyclo-hexyl-carbodiimide (DCC) (1.1 g, 1.5 eq.), 4-Dimethylaminopyridine (DMAP) (0.1 g, 0.1 eq.) in dichloromethane (10 mL) was stirred at room temperature until the complete conversion of starting materials indicated by TLC. Then the reaction mixture was filtered, and filtrate was stirred with saturated NaHCO₃ solution. Then the organic layer was separated, dried over anhydrous Na₂SO₄ and concentrated under vacuum. The obtained crude product was purified by column chromatography on 100-200 silica gel with 10-20% ethylacetate in n-hexane as eluent to get desired product in 55% yield.

Characterisation (Analytical data): ¹H NMR (400 MHz, DMSO-d₆) δ 9.26 (dd, J=2.2, 0.8 Hz, 1H), 8.93-8.91 (m, 1H), 8.47 (dt, J=8.0, 1.8 Hz, 1H), 7.69-7.65(m, 2H), 7.54 (d, J=2.0 Hz, 1H), 7.48 (d, J=8.3 Hz, 1H), 3.87 (s, 3H), 0.78 (s, 9H), 0.16 (s, 6H).

### Step-2: Synthesis of 2-hydroxy-5-(methoxycarbonyl)phenyl nicotinate

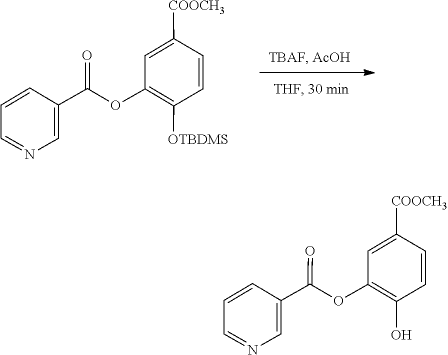 [SMILES: COC(=O)c1ccc(O[Si](C)(C)C(C)(C)C)c(OC(=O)c2cccnc2)c1 ; COC(=O)c1ccc(O)c(OC(=O)c2cccnc2)c1]

A mixture of 2-((tert-butyldimethylsilyl)oxy)-5-(methoxycarbonyl)phenyl nicotinate (0.3 g, 1 eq.) (from step-1 above) in THF (10 mL) was treated with acetic acid (0.3 mL) and 1M solution of tetrabutylammonium fluoride (0.41 g, 2 eq.) at below 5° C. temperature until the reaction completion (30 min) indicated by TLC. Then the reaction mixture was poured into ice cold water and extracted with ethyl acetate (2×20 mL). Combined organic layers were dried over anhydrous Na₂SO₄ and evaporated under vacuum. Then the product was purified by column chromatography on silica gel with 15-25% ethylacetate in n-hexane as eluent to afford target compound in 65% yield.

Characterisation (Analytical data): ¹H NMR (400 MHz, DMSO-d₆) δ 10.84 (s, 1H), 9.29-9.24 (m, 1H), 8.91 (dd, J=4.8, 1.6 Hz, 1H), 8.47 (dt, J=7.9, 1.9 Hz, 1H), 7.80 (dd, J=6.5, 2.1 Hz, 2H), 7.66 (dd, J=7.8, 4.9 Hz, 1H), 7.09 (d, J=9.1 Hz, 1H), 3.82 (s, 3H).

### Example 6

### Synthesis of a mixture of 3-hydroxy-5-(methoxycarbonyl)phenyl nicotinate & 5-(methoxycarbonyl)-1,3-phenylene dinicotinate: (Process of Synthesis Same as in Example 3)

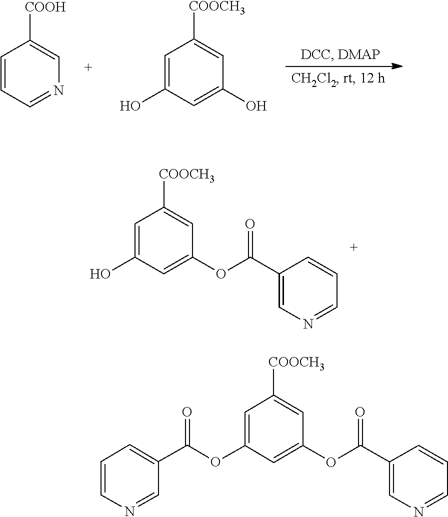 [SMILES: O=C(O)c1cccnc1 ; COC(=O)c1cc(O)cc(O)c1 ; COC(=O)c1cc(O)cc(OC(=O)c2cccnc2)c1 ; COC(=O)c1cc(OC(=O)c2cccnc2)cc(OC(=O)c2cccnc2)c1]

Both the compounds from the mixture were separated and characterised as in below table-1:

| 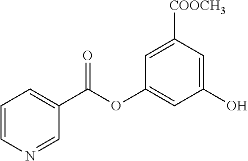 [SMILES: COC(=O)c1cc(O)cc(OC(=O)c2cccnc2)c1] | 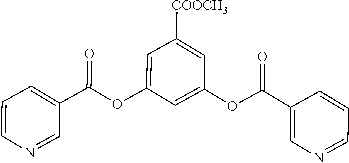 [SMILES: COC(=O)c1cc(OC(=O)c2cccnc2)cc(OC(=O)c2cccnc2)c1] |
|---|---|
| 3-hydroxy-5- | 5-(methoxycarbonyl)-1,3-phenylene |
| (methoxycarbonyl)phenyl nicotinate | dinicotinate |
| Characterization (Analytical Data): | Characterization (Analytical Data): |
| Yield: 30%; ¹H NMR (400 MHz, | Yield: 50%; Mp: 138-139° C.; ¹H NMR (400 |
| DMSO-d₆) δ 10.29 (s, 1H), 9.26 (dd, J = | MHz, DMSO-d₆) δ 9.34 (dd, J = 2.2, 0.8 Hz, |
| 2.2, 0.8 Hz, 1H), 8.91 (dd, J = 4.8, | 2H), 8.97 (dd, J = 4.8, 1.7 Hz, 2H), 8.54 (dt, J = |
| 1.7 Hz, 1H), 8.47 (dt, J = 8.0, 1.9 Hz, | 8.0, 1.9 Hz, 2H), 7.98 (d, J = 2.2 Hz, 2H), 7.83 |
| 1H), 7.68-7.64 (m, 1H), 7.35-7.32 (m, | (t, J = 2.2 Hz, 1H), 7.74-7.71 (m, 2H), 3.96 (s, |
| 2H), 7.02 (t, J = 2.2 Hz, 1H), 3.85 (s, | 3H); ¹³C NMR (100 MHz, DMSO-d₆) δ 165.2, |
| 3H). | 163.8, 154.8, 151.4, 151.1, 138.1, 132.3, 125.3, |
| | 124.5, 121.6, 121.2, 53.2. IR (KBr, v\tilde{ }, cm⁻¹) |
| | 3038, 1738, 1735, 1590, 1404, 1279, 1139, |
| | 1095, 1019, 892, 767, 730, 620. |

### Example 7

### Synthesis of 4-((benzyloxy)carbonyl)phenyl nicotinate

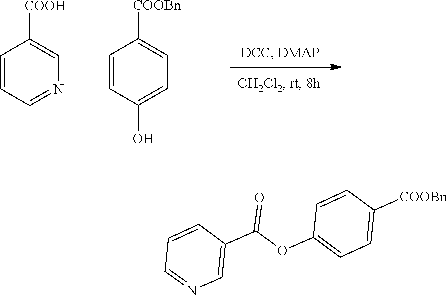

A solution of phenolic benzyl ester derivative (1 eq.) in dichloromethane (10 wv), charged nicotinic acid (1.5 eq.), DCC (1.5 eq.) and DMAP (0.1 eq.). The reaction mixture was stirred at room temperature for 8-15 hr. Reaction progress was observed by thin layer chromatography. After completion of the reaction, reaction mixture was filtered and filtrate (dichloromethane layer) was transferred to saturated NaHCO₃ solution. Then the organic layer separated and concentrated under vacuum. Obtained crude product was purified by column chromatography on 100-200 silica gel with 15-30% ethylacetate in n-hexane as eluents to get desired products in 50-85% yields.

Characterisation (Analytical data): Yield: 76%; ¹H NMR (400 MHz, DMSO-d₆) δ 9.28 (dd, J=2.2, 0.7 Hz, 1H), 8.91 (dd, J=4.8, 1.7 Hz, 1H), 8.48 (dt, J=8.0, 1.9 Hz, 1H), 8.12 (d, J=8.8 Hz, 2H), 7.68-7.64 (m, 1H), 7.52 (d, J=8.7 Hz, 2H), 7.49 (s, 2H), 7.44-7.34 (m, 3H), 5.38 (s, 2H); ¹³C NMR (100 MHz, DMSO-d₆) δ 165.3, 163.7, 154.8, 154.6, 151.1, 138.0, 136.5, 131.4, 129.0, 128.6, 128.5, 128.0, 125.3, 124.5, 122.9, 66.8.

### Example 8

### Synthesis of 4-((benzyloxy)carbonyl)-2-methoxyphenyl nicotinate

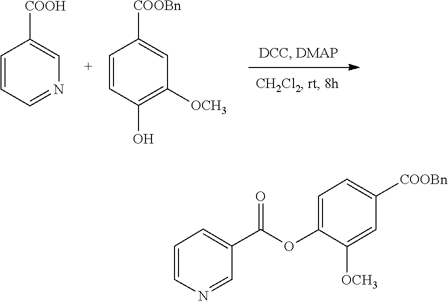 [0121] Same process of synthesis as in example 7 is used for preparation of 4-((benzyloxy)carbonyl)-2-methoxyphenyl nicotinate.

Characterisation (Analytical data): Yield: 82%; ¹H NMR (400 MHz, DMSO-d₆) δ 9.31 (dd, J=2.2, 0.9 Hz, 1H), 8.97 (dd, J=4.9, 1.7 Hz, 1H), 8.52 (dt, J=8.0, 1.9 Hz, 1H), 7.76 (s, 1H), 7.76-7.68 (m, 2H), 7.57-7.36 (m, 6H), 5.44 (s, 2H), 3.90 (s, 3H); ¹³C NMR (100 MHz, DMSO-d₆) δ 165.4, 163.2, 155.0, 151.4, 151.0, 143.5, 138.1, 136.5, 129.2, 129.0, 128.6, 128.4, 124.9, 124.7, 123.9, 122.7, 113.6, 66.9, 56.6.

### Example 9

### Synthesis of 4-((benzyloxy)carbonyl)-2,6-dimethoxyphenyl nicotinate

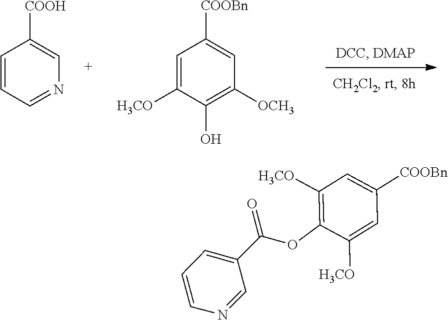 [0124] Same process of synthesis as in example 7 is used for preparation of 4-((benzyloxy)carbonyl)-2,6-dimethoxyphenyl nicotinate.

Characterisation (Analytical data): Yield: 80%; Mp: 111-112° C.; ¹H NMR (400 MHz, DMSO-d₆) δ 9.29 (d, J=1.6 Hz, 1H), 8.95 (dd, J=4.8, 1.5 Hz, 1H), 8.50 (dt, J=8.0, 1.8 Hz, 1H), 7.68 (dd, J=7.8, 4.9 Hz, 1H), 7.52 (d, J=7.1 Hz, 2H), 7.45-7.36 (m, 5H), 5.44 (s, 2H), 3.86 (s, 6H); ¹³C NMR (100 MHz, DMSO-d₆) δ 165.4, 162.7, 155.1, 152.4, 151.0, 138.1, 136.6, 132.1, 129.0, 128.7, 128.6, 128.4, 124.7, 124.6, 106.4, 67.0, 56.8.

### Example 10

### Synthesis of 2-(benzyloxy)-5-((benzyloxy)carbonyl)phenyl nicotinate

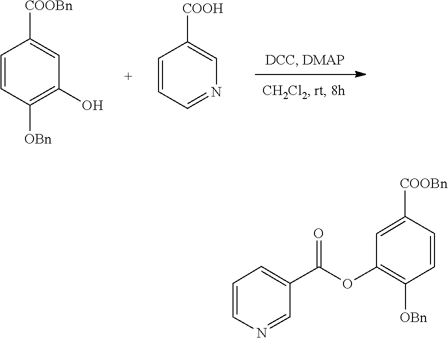 [0127] Same process of synthesis as in example 7 is used for preparation of 2-(benzyloxy)-5-((benzyloxy)carbonyl)phenyl nicotinate.

Characterisation (Analytical data): Yield: 76%; Mp: 70-72° C.; ¹H NMR (400 MHz, CDCl₃) δ 9.31 (d, J=1.5 Hz, 1H), 8.77 (dd, J=4.9, 1.7 Hz, 1H), 8.35 (dt, J=8.0, 2.0 Hz, 1H), 7.92 (dd, J=8.6, 2.1 Hz, 1H), 7.84 (d, J=2.1 Hz, 1H), 7.39-7.26 (m, 6H), 7.23-7.16 (m, 5H), 6.99 (d, J=8.7 Hz, 1H), 5.27 (s, 2H), 5.09 (s, 2H); ¹³C NMR (100 MHz, CDCl₃) δ 165.3, 163.3, 154.0, 151.5, 139.6, 137.7, 136.0, 135.7, 129.5, 128.6, 128.2, 128.1, 127.1, 126.9, 124.5, 123.4, 123.2, 122.8, 113.2, 70.7, 66.8.

### Example 11

### Synthesis of 2-(benzyloxy)-4-((benzyloxy)carbonyl)phenyl nicotinate

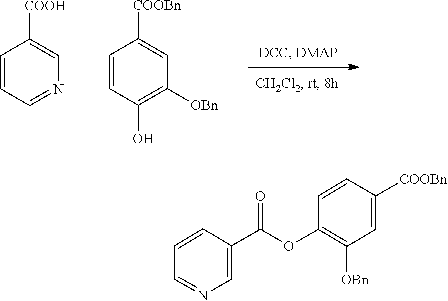 [0130] Same process of synthesis as in example 7 is used for preparation of 2-(benzyloxy)-4-((benzyloxy)carbonyl)phenyl nicotinate.

Characterisation (Analytical data): Yield: 82%; ¹H NMR (400 MHz, CDCl₃) δ 9.30 (d, J=2.8 Hz, 1H), 8.77 (dd, J=4.9, 1.7 Hz, 1H), 8.34 (dt, J=8.0, 2.0 Hz, 1H), 7.73-7.69 (m, 2H), 7.39-7.28 (m, 6H), 7.23-7.16 (m, 6H), 5.29 (s, 2H), 5.07 (s, 2H); ¹³C NMR (100 MHz, CDCl₃) δ 165.6, 163.0, 154.1, 151.5, 150.2, 144.0, 137.7, 136.0, 135.9, 129.1, 128.6, 128.5, 128.3, 128.2, 128.1, 127.1, 125.2, 123.4, 123.2, 122.8, 115.3, 70.9, 67.0.

### Example 12

### Synthesis of a mixture of 4-((benzyloxy)carbonyl)-2-hydroxyphenyl nicotinate & 4-((benzyloxy)carbonyl)-1,2-phenylene dinicotinate

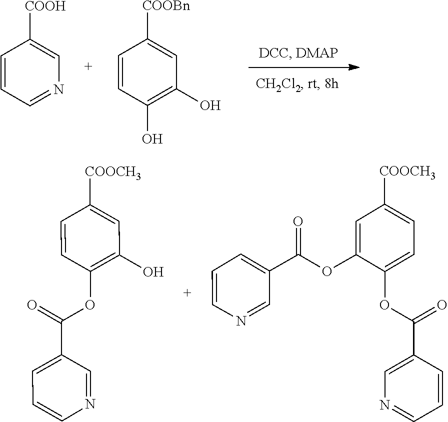 [0133] Same process of synthesis as in example 7 is used for preparation of the above mixture.

Both the compounds from the mixture were separated and characterised as in below table-2:

| 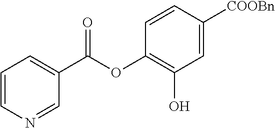 | 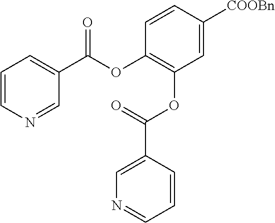 |
|---|---|
| 4-((benzyloxy)carbonyl)-2- | 4-((benzyloxy)carbonyl)-1,2-phenylene |
| hydroxyphenyl nicotinate. | dinicotinate. |
| Characterization (Analytical Data): | Characterization (Analytical Data): |
| Yield: 35%; ¹H NMR (400 MHz, | Yield: 46%; ¹H NMR (400 MHz, DMSO- |
| DMSO-d₆) δ 10.84 (s, 1H), 9.26 (dd, J = | d₆) δ 9.13-9.11 (m, 2H), 8.83 (td, J = 4.9, |
| 2.2, 0.7 Hz, 1H), 8.90 (dd, J = 4.8, 1.7 | 1.7 Hz, 2H), 8.36-8.32 (m, 2H), 8.20 (d, J = |
| Hz, 1H), 8.46 (dt, J = 8.0, 1.9 Hz, 1H), | 2.0 Hz, 1H), 8.10 (dd, J = 8.5, 2.1 Hz, |
| 7.86-7.82 (m, 2H), 7.67-7.63 (m, 1H), | 1H), 7.77 (d, J = 8.5 Hz, 1H), 7.58-7.50 |
| 7.50-7.32 (m, 5H), 7.11 (d, J = 9.1 Hz, | (m, 4H), 7.45-7.35 (m, 3H), 5.41 (s, 2H). |
| 1H), 5.32 (s, 2H). | |

### Example 13

### Synthesis of a mixture of 3-((benzyloxy)carbonyl)-5-hydroxyphenyl nicotinate & 5-((benzyloxy)carbonyl)-1,3-phenylene dinicotinate

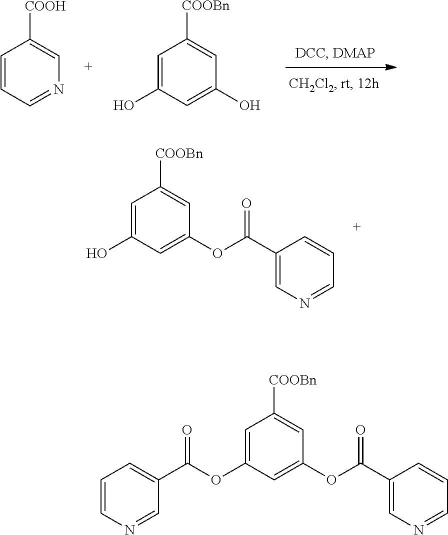 [0136] Same process of synthesis as in example 7 is used for preparation of the above mixture.

Both the compounds from the mixture were separated and characterised as in below table-3:

| 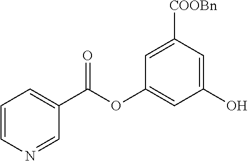 | 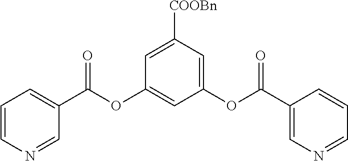 |
|---|---|
| 3-((benzyloxy)carbonyl)-5- | 5-((benzyloxy)carbonyl)-1,3-phenylene |
| hydroxyphenyl nicotinate | dinicotinate |
| Characterization (Analytical Data): | Characterization (Analytical Data): |
| Yield: 30%; ¹H NMR (400 MHz, | Yield: 50%; ¹H NMR (400 MHz, DMSO- |
| DMSO-d₆) δ 10.29 (s, 1H), 9.27-9.23 (m, | d₆) δ 9.28 (d, J = 1.5 Hz, 2H), 8.91 (dd, J = |
| 1H), 8.89 (dd, J = 4.8, 1.7 Hz, 1H), 8.44 | 4.8, 1.7 Hz, 2H), 8.49 (dt, J = 8.0, 1.9 Hz, |
| (dt, J = 8.0, 1.9 Hz, 1H), 7.66-7.60 (m, | 2H), 7.96 (d, J = 2.2 Hz, 2H), 7.79 (t, J = |
| 1H), 7.50-7.32 (m, 7H), 7.04 (t, J = 2.2 | 2.2 Hz, 1H), 7.69-7.63 (m, 2H), 7.52-7.48 |
| Hz, 1H), 5.35 (s, 2H). | (m, 2H), 7.44-7.33 (m, 3H), 5.41 (s, 2H). |

### Example 14

### Synthesis of 2-(benzyloxy)-5-((benzyloxy)carbonyl)-3-hydroxyphenyl nicotinate

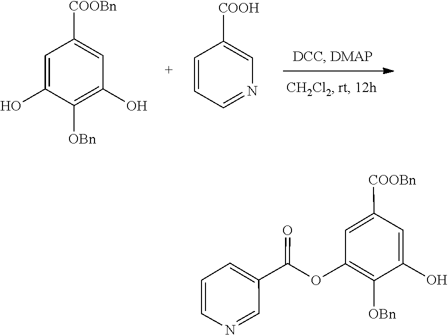 [0139] Same process of synthesis as in example 7 is used for preparation of 2-(benzyloxy)-5-((benzyloxy)carbonyl)-3-hydroxyphenyl nicotinate.

Characterisation (Analytical data): Yield: 56%; ¹H NMR (400 MHz, CDCl₃) δ 9.25 (dd, J=2.2, 0.8 Hz, 1H), 8.78 (dd, J=4.9, 1.7 Hz, 1H), 8.29 (dt, J=8.0, 1.9 Hz, 1H), 7.54 (d, J=2.0 Hz, 1H), 7.43 (d, J=2.0 Hz, 1H), 7.40-7.27 (m, 6H), 7.20-7.15 (m, 5H), 5.25 (s, 2H), 5.00 (s, 2H); ¹³C NMR (100 MHz, CDCl₃) δ 165.1, 163.1, 154.1, 151.3, 150.2, 142.9, 142.2, 137.8, 135.9, 135.7, 128.8, 128.7, 128.6, 128.4, 128.3, 128.2, 126.3, 124.9, 123.6, 116.6, 115.5, 76.2, 67.0.

### Example 15

### Synthesis of 2,6-bis(benzyloxy)-4-((benzyloxy)carbonyl)phenyl nicotinate

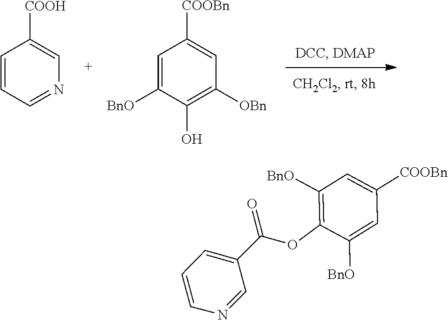 [0142] Same process of synthesis as in example 7 is used for preparation of 2,6-bis(benzyloxy)-4-((benzyloxy)carbonyl)phenyl nicotinate.

Characterisation (Analytical data): Yield: 78%; ¹H NMR (400 MHz, DMSO-d₆) δ 9.27 (dd, J=2.2, 0.8 Hz, 1H), 8.92 (dd, J=4.8, 1.7 Hz, 1H), 8.48 (dt, J=8.0, 1.9 Hz, 1H), 7.66-7.63 (m, 1H), 7.48 (s, 2H), 7.45-7.37 (m, 5H), 7.35-7.30 (m, 5H), 7.29-7.25 (m, 5H), 5.38 (s, 2H), 5.25 (s, 4H); ¹³C NMR (100 MHz, DMSO-d₆) δ 165.2, 163.0, 155.1, 151.4, 150.9, 138.0, 136.8, 136.5, 133.2, 129.0, 128.8, 128.6, 128.4, 128.3, 128.2, 127.5, 124.7, 108.4, 70.8, 66.9.

### Example 16

### Synthesis of 4-((allyloxy)carbonyl)phenyl nicotinate

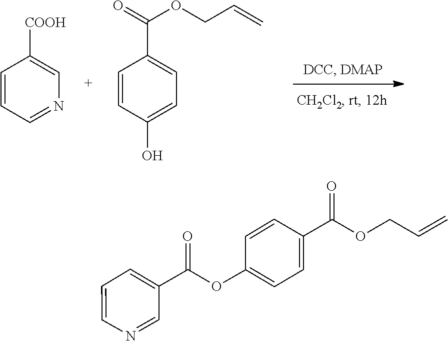 [SMILES: O=C(O)c1cccnc1 ; C=CCOC(=O)c1ccc(O)cc1 ; C=CCOC(=O)c1ccc(OC(=O)c2cccnc2)cc1] [0145] Same process of synthesis as in example 7 is used for preparation of 4-((allyloxy)carbonyl)phenyl nicotinate, wherein instead of phenolic benzyl ester derivative, phenolic allyl ester derivative was used.

Characterisation (Analytical data): Yield: 75%; ¹H NMR (400 MHz, DMSO-d₆) δ 9.28 (dd, J=2.2, 0.9 Hz, 1H), 8.92 (dd, J=4.8, 1.7 Hz, 1H), 8.49 (dt, J=8.0, 1.8 Hz, 1H), 8.11 (d, J=8.7 Hz, 2H), 7.68-7.65 (m, 1H), 7.52 (d, J=8.7 Hz, 2H), 6.12-6.02 (m,1H), 5.43 (dq, J=17.3, 1.7 Hz, 1H), 5.30 (dq, J=10.5, 1.4 Hz, 1H), 4.84 (dt, J=5.4, 1.5 Hz, 2H); ¹³C NMR (100 MHz, DMSO-d₆) δ 165.1, 163.7, 154.8, 154.6, 151.1, 138.1, 133.0, 131.4, 127.9, 125.3, 124.5, 122.9, 118.4, 65.7.

We claim:

1. A substituted pyridine carboxylic acid derivative compound of Formula I: 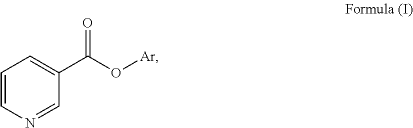
or a pharmaceutically acceptable salt, isomer, ester, prodrug or solvate thereof, wherein Ar is selected from the following compounds:

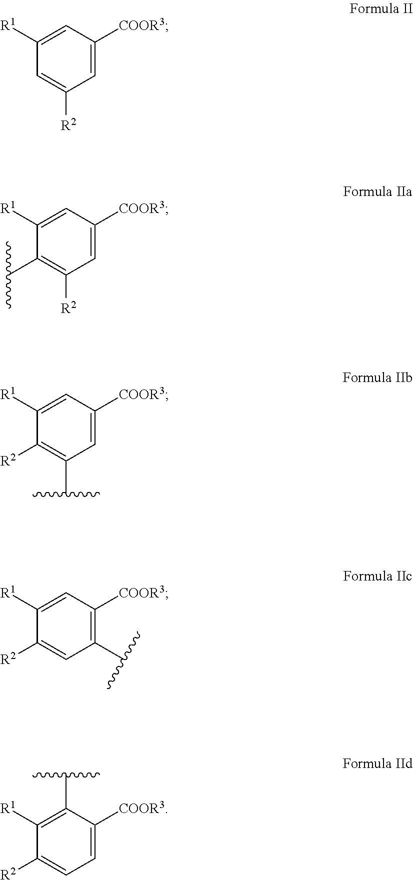

2. The compound as claimed in claim 1, wherein in the compound of Formula II, IIa, IIb, IIc and IId, the groups,
R¹ & R² represents one or more independent substitutions in the benzene moiety selected from the group comprising of —H, —OH, alkyl, alkenyl, alkynyl, halogen, cycloalkyl, cyano, alkoxy, carboxylic derivative, amine, aryl or any other suitable aliphatic group and can be interchangeably positioned; and
R³ represents one or more independent substitutions in the benzene moiety selected from the group comprising of —H, -alkyl, -amine, phenyl, benzene or any other suitable aliphatic or aromatic group.

3. The compound as claimed in claim 1, wherein in the compound of Formula II IIa, IIb, IIc and IId, the groups R1, R2 could be the same or different functional moieties selected from the functional groups comprising of —H, —OH, alkyl, alkenyl, alkynyl, halogen, cycloalkyl, cyano, alkoxy, carboxylic derivative, amine, aryl or any other suitable aliphatic group and can be suitably interchanged in position.

4. A pharmaceutical compositions comprising of a compound of Formula I: 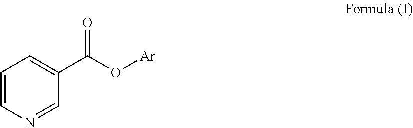
or a pharmaceutically acceptable salt, isomer, ester, prodrug or solvate thereof and a pharmaceutically acceptable carrier or excipient.

5. The pharmaceutical composition as claimed in claim 4, wherein the said composition is useful for treatment of cholinergic receptor mediated diseases, for management of hyperlipidemia and hypercholesterolemia and associated cardiovascular diseases, diabetes, nutritional disorders, inflammation, proliferative disease, skin disorders and other metabolic disorders.

6. The pharmaceutical composition as claimed in claim 4, wherein the composition comprises a pharmaceutically effective amount of the compound of formula (I) or a pharmaceutically acceptable salt, isomer, ester, prodrug or solvate thereof and a pharmaceutically acceptable carrier or excipient.

7. The pharmaceutical composition as claimed in claim 4, wherein the said composition comprising of a pharmaceutically effective amount of the compound of formula (I) or a pharmaceutically acceptable salt, isomer, ester, prodrug or solvate thereof and a pharmaceutically acceptable carrier or excipient in combination with another therapeutic agent.

8. The pharmaceutical composition as claimed in claim 4, wherein the said composition is formulated as solid dosage forms selected from the group comprising of tablets, suspension tablets, bite suspension tablets, rapid dispersion tablets, chewable tablets, melt tablets, effervescent tablets, bi-layer tablets, caplets, capsules, powders, lozenges, sachets, cachets, troches, pellets, granules, micro-granules, encapsulated micro-granules, powder aerosol formulations, or any other solid dosage form reasonably adapted for oral administration; as liquid dosage form selected from the group comprising of solutions, suspension, elixirs, syrups, liquid aerosol formulations and as topical dosage from selected from ointment, gel, emulsion, creams, sprays and dispersions; or as rectal, intravenous of intramuscular administration.

9. A process for preparation of a compound of Formula-I, comprising of following steps:
a. reacting a solution of nicotinic acid derivative with corresponding solution of hydroxy substituted benzoate derivatives;
b. stirring the resulting mixture of step a) above room temperature or elevated temperatures for sufficient time;
c. monitoring the reaction for completion, confirmed by TLC or any other suitable known analytical technique;
d. followed by cooling the reaction mixture of step b) and quenching with solvent;
e. then solvent was completely removed under rota evaporator or by using precipitation technique from step d) mixture and thereafter the residue was dissolved in alcohol or any other organic solvent and silica gel was added to it followed by drying to get dried slurry;
f. then the slurry obtained from step e) was loaded onto column and eluted using suitable solvent to get the pyridine derivative compound with good yield; and
g. finally the isolated compound obtained which may optionally be subjected to crystallization by dissolving in organic solvent and adding a good crystallization solvent, followed by cooling to get the crystal and separating the crystal from mother liquor and drying.

10. The process as claimed in claim 9, wherein in the process steps for preparation of compound of formula (I) can be used for the next step with or without any additional purification steps.

11. A method for management of diabetes by administering a substituted pyridine carboxylic acid derivative compound of Formula I: 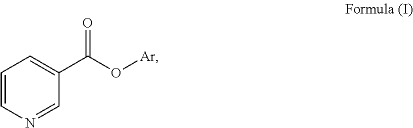
or a pharmaceutically acceptable salt, isomer, ester, prodrug or solvate thereof, wherein Ar is selected from the following compounds:

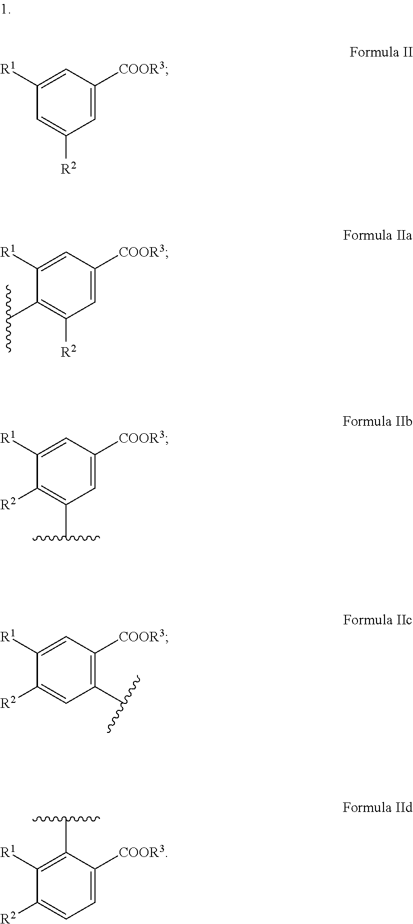

12. The method as claimed in claim 11, wherein the compound of Formula-I can be administered as a composition comprising of a pharmaceutically effective amount of the compound of formula (I) or a pharmaceutically acceptable salt, isomer, ester, prodrug or solvate thereof and a pharmaceutically acceptable carrier or excipient.

13. A method for management of cholinergic receptor mediated diseases, hyperlipidemia and hypercholesterolemia and associated cardiovascular diseases, nutritional disorders, inflammation, proliferative disease, skin disorders and other metabolic disorders by administering a substituted pyridine carboxylic acid derivative compound of Formula I:

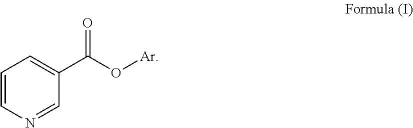

14. The method as claimed in claim 13, wherein in the substituted pyridine carboxylic acid derivative compound of Formula I can be administered as a composition comprising of a pharmaceutically effective amount of the compound of formula (I) or a pharmaceutically acceptable salt, isomer, ester, prodrug or solvate thereof and a pharmaceutically acceptable carrier or excipient.

15. Use of a substituted pyridine carboxylic acid derivative compound of Formula I: 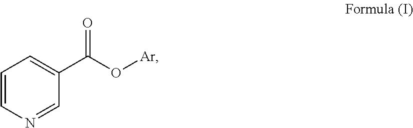
or a pharmaceutically acceptable salt, isomer, ester, prodrug or solvate thereof, wherein Ar is selected from the following compounds: 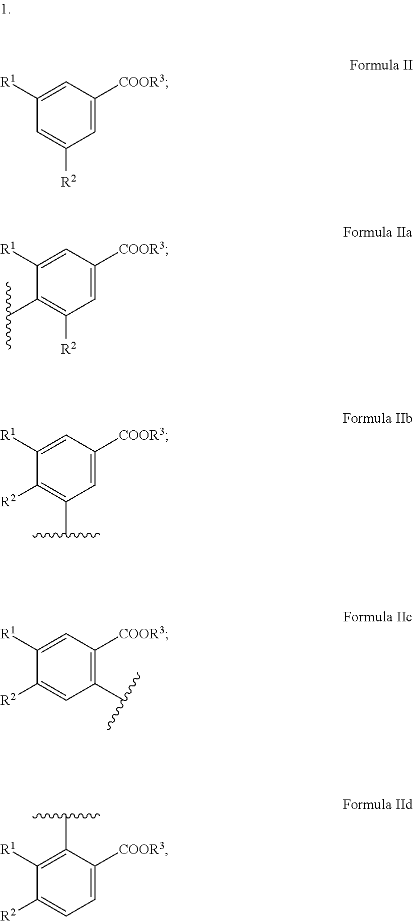
for treatment of cholinergic receptor mediated diseases, for management of hyperlipidemia and hypercholesterolemia and associated cardiovascular diseases, diabetes, nutritional disorders, inflammation, proliferative disease, skin disorders and other metabolic disorders.
